(19) United States
(12) Patent Application Publication
(10) Pub. No.: US 2024/0350743 A1
(43) Pub. Date: Oct. 24, 2024

(54) Cap for medicament delivery device

(71) Applicant: SHL Medical AG, Zug (CH)

(72) Inventor: Lucas Carpenter, Taoyuan City (TW)

(21) Appl. No.: 18/683,591

(22) PCT Filed: Aug. 18, 2022
(86) PCT No.: PCT/EP2022/073134

§ 371 Date: Feb. 14, 2024

(30) Foreign Application Priority Data
Aug. 24, 2021 (EP) 21192817.1

(51) Int. Cl. A61M 5/32

(52) U.S. Cl. CPC A61M 5/3202

(57) ABSTRACT

The present disclosure provides a cap assembly (1) for a medicament delivery device (6), the cap assembly comprising a tubular body (4) and a first lid (2), wherein the tubular body (4) comprises a proximal opening (7) for receiving a proximal part of a medicament delivery device, wherein the tubular body (4) is expandable in a longitudinal direction; and wherein the cap assembly is configured to change shape from a first, not expanded, configuration for protecting a medicament delivery member of the medicament delivery device prior to use to a second, expanded, configuration for storing the medicament delivery device (6) after use.

### TECHNICAL FIELD

The present invention relates to cap assemblies for medicament delivery devices. More particularly, the present invention relates to a cap assembly that is configured to change shape from a first, not expanded, configuration for protecting the medicament delivery member of the medicament delivery device prior to use to a second, expanded, configuration for storing the used medicament delivery device after use.

### BACKGROUND

In hospitals or other medical facilities, containers for receiving articles such as sharps (used syringes, etc.) are known and some of these are arranged so that they can be locked closed prior to incineration. Sharps containers in general provide a sealed environment which will reduce the possibility of contact by the handler with the secured contents. Nevertheless, the applicant has appreciated that there is still further scope for improvement of the packaging of medicament delivery devices with sharps features such as those described in WO2009/053693.

Medicament delivery devices or on-body devices, such as hypodermic syringes, with needles, auto-injectors, safety syringes, pen-injectors are generally known for the self-administration of a medicament by patients without formal medical training occurs. As just one example, those patients suffering from diabetes may require repeated injections of insulin. Other patients may require regular injections of other types of medicaments, such as a growth hormone or biological medicaments. Therefore, to facilitate for the patients, there is a demand to design medicament delivery devices with multiple automatic functions, predetermined dosage and assembled with a medicament container. Some of the medicament delivery devices are single use devices and disposable after use, other medicament delivery devices may have reusable parts, and only the sharps or contaminated parts such as needles, tubes or nozzles are disposed after use. But in a private or non-medical environment a safe storage of sharps is also advantageous.

EP 2 298 224 A2 describes an example of a shipping container integrating a sharps disposal container with a new product storage container for shipping. The storage container stores new product. A sharps disposal container receives used sharps. One of the storage container and the sharps disposal container is disposed within the other of the storage container and the sharps disposal container for shipping. A lid is adapted to be connected to the outermost container of the sharps disposal container and the storage container for shipping and to a top of the sharps disposal container for receiving used sharps.

### BRIEF DESCRIPTION

The present disclosure is applicable to a number of medicament delivery devices, including, but not limited to, devices that automatically, semi-automatically or manually deliver one or more doses of medicament through injection (needle and needleless). Further, the disclosure is applicable to a number of packaging containers and packaging materials.

As indicated, a general object of the present disclosure is to provide a safety cap for a medicament delivery device which is transformable into a disposal container for disposable medicament delivery devices.

The invention is defined by the appended claims, to which reference should now be made.

An aspect concerns a cap assembly for a medicament delivery device, the cap assembly comprising a tubular body and a first lid, wherein the tubular body comprises a proximal opening for receiving a proximal part of a medicament delivery device, wherein the tubular body is expandable in a longitudinal direction; and wherein the cap assembly is configured to change shape from a first, not expanded, configuration for protecting a medicament delivery member of the medicament delivery device prior to use to a second, expanded, configuration for storing the medicament delivery device after use.

Optionally the cap assembly further comprises a second lid which is arranged, when in the first configuration, between the tubular body and the first lid.

Optionally the tubular body comprises a distal opening, and at a distal end of the cap assembly, a first ring configured to fasten the second lid, and wherein the tubular body further comprises at a proximal end a second ring.

Optionally, the tubular body is a cap for the medicament delivery device wherein the tubular body forms a sleeve around a proximal end of the medicament delivery device, wherein the distal opening has a smaller diameter than the proximal opening; and wherein the distal opening further comprises an inner rim providing an abutting shoulder for the proximal end of the medicament delivery device.

Optionally the ring further comprises a locking mechanism configured to lock the tubular body in a retracted state when in the first configuration.

Optionally when the locking mechanism is unlocked the tubular body is configured to expand along an axis L so as to enclose the medicament delivery device (6) inside the tubular body.

Optionally the locking mechanism comprises a longitudinal seal extending from the first ring to the second ring wherein the seal is broken when the locking mechanism is actuated.

Optionally the locking mechanism comprises a slit arranged on the second ring wherein the slit is configured to receive a protrusion, wherein the protrusion is attached to an arm, which arm is attached to the first ring and the arm extends from the first ring to the second ring.

Optionally the protrusion extends beyond the slit in the distal direction, the protrusion also extends circumferentially beyond the circumferential width of the arm, thereby the protrusion defines a circumferentially extending part, wherein the circumferentially extending part of the protrusion is configured to move beyond the opening of the slit when the first ring is moved in a first direction, so that the circumferentially extending part of the protrusion inhibits a movement in the proximal direction of the tubular body when the tubular body is locked in a contracted state.

Optionally when the first ring being moved in a second direction, the second direction being opposite to the first direction, then the the circumferentially extending part of the protrusion is configured to align with the slit such that the protrusion and the circumferentially part passes through the slit, such that the protrusion can move in the proximal direction, thereby allowing the tubular body to move in the proximal direction.

Optionally the tubular body comprises an expandable structure in the form of a folded accordion, or a rolled-up structure, or an interlocked nested structure.

Optionally, the tubular body has a cross sectional shape which snugly fits the shape of the medicament delivery device which is introduced into the tubular body.

A second aspect concerns a method for transforming the cap assembly according to any of the preceding claims comprising the steps of: removing the cap assembly from the medicament delivery device; removing the first lid; inserting the medicament delivery device into a proximal opening after the medicament delivery device has been used; unlocking the locking mechanism; expanding the tubular body over the whole length of the medicament delivery device; and closing the proximal opening with the first lid.

Optionally the method further comprises the steps of: removing a second lid; and closing a distal opening with a second lid.

A third aspect concerns a medicament delivery device comprising the cap assembly as described above.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component. The circumferential direction describes a direction extending around the axis, so around the circumference of a device or component, and the radial direction extends perpendicular to the axis. Similarly, the terms “transverse”, “transversal” and “transversally” refer to a direction generally perpendicular to the longitudinal direction.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

The term automatic in conjunction with medical injection device means that, the injector device is able to perform the injection without the user of the device delivering the force needed to expel the medicament during delivery. The force is delivered automatically by a resilient force like a spring or an electric motor or any other suitable means for providing the delivery force.

### BRIEF DESCRIPTION OF THE DRAWINGS

Embodiments of the invention will now be described by way of example only and with reference to the accompanying drawings, in which:

FIG. 1 shows a perspective view of the cap assembly 1 in an initial state.

FIG. 2 shows a perspective view of the cap assembly 1 of FIG. 1 with the first lid 2 removed.

FIG. 3 shows a perspective view of the cap assembly 1 in an exploded view with a medicament delivery device.

FIG. 4 shows a perspective view of the cap assembly 1 in an expanded state.

FIG. 5 shows the tubular body 4 during expansion over a medicament delivery device 6.

FIG. 6 shows the full expansion of the tubular body before the first lid 2 is attached.

FIG. 7 shows the cap assembly 1 fully expanded over the medicament delivery device with the first lid 2 closing the sharps container.

FIG. 8 shows the cap assembly 1 and the medicament delivery device 6 in an initial state.

FIG. 9 shows the cap assembly 1 pulled off the medicament delivery device.

FIG. 10 shows the cap assembly 1 on the medicament delivery device 6 disassembled into the first lid 2 and the tubular body 4.

FIG. 11 shows unlocking of the tubular body 4 seated on the medicament delivery device 6.

FIG. 12 shows a perspective view of the cap assembly, with a cross-section view of the cap to show the locking mechanism.

FIG. 13 shows a further perspective view of FIG. 12.

### DETAILED DESCRIPTION

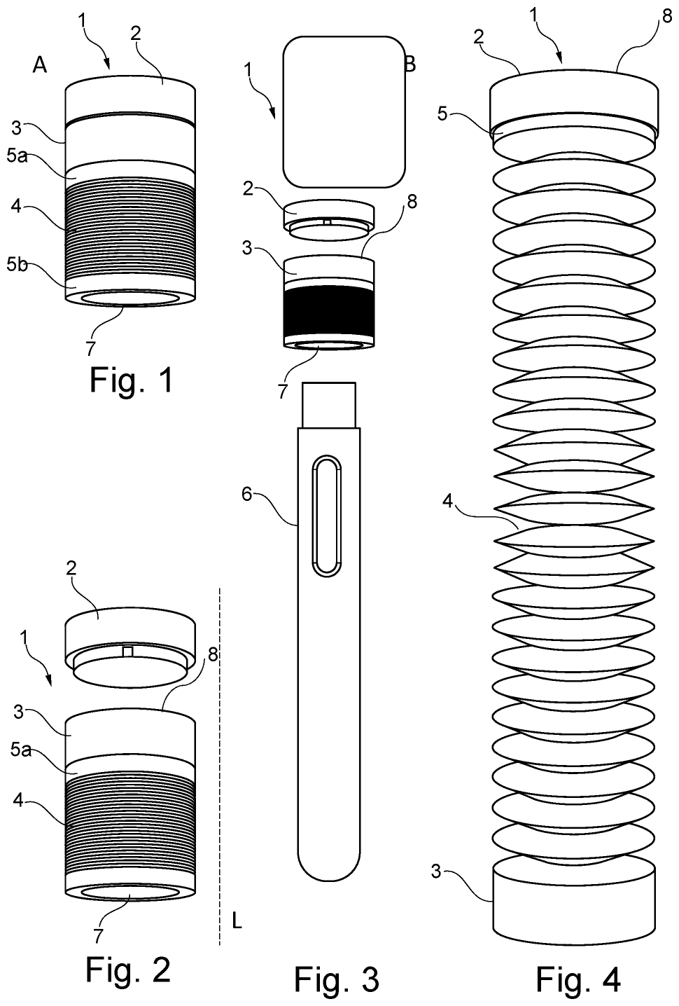

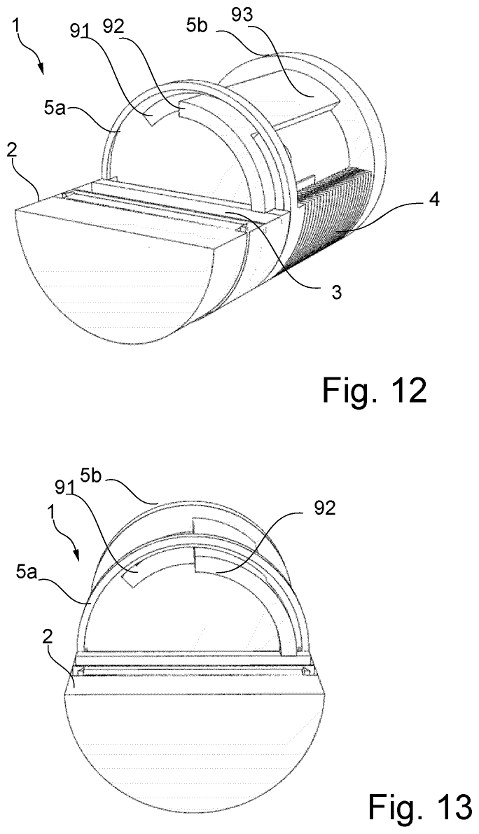

As shown in FIGS. 1 to 4, the cap assembly 1 having a longitudinal compact shape along an axis L and comprising at least two parts 2,4 which are attached to each other. For instance, the cap assembly 1 comprises an expandable hollow tubular body 4 having an opening 7 on its proximal side configured to receive the medicament delivery device 6 within the hollow opening 7. The tubular body 4 is mounted like a sleeve onto the proximal part of medicament delivery device 6, and a first lid 2 is attached to the distal end of the cap assembly, thereby closing the opening 8 of the tubular body 4, which further comprises a locking mechanism as shown in FIGS. 12 and 13, configured to prevent the tubular body 4 to expand accidentally. The tubular body 4 has on each end a ring, namely a first ring 5a and a second ring 5b, wherein the second ring is configured to provide a locking and/or mounting mechanism which will be described in more detail further below.

The cap assembly 1 optionally further comprises a second, closing, lid 3, mounted between the tubular body 4 and the first lid 2 when in the initial position. Instead of an inner rim of the opening 8 comprising a shoulder for the proximal end of the medicament delivery device 6 to abut on, the second lid 3 closes the opening 8.

The cap assembly 1 is expandable from an initial, retracted state A into a final, expanded state B, configured to confine the medicament delivery device 6, thus providing a sharps container for the used and disposable medicament delivery device 6 when in the final state B.

In this example the tubular body 4 has a cylindrical shape with a circular opening on the proximal end, namely the proximal opening 7, and another circular opening on the distal end, namely the distal opening 8. In other words, the tubular body 4 has a circular cross section and a hollow cylindrical shape. In another example the tubular body has only one opening, namely the proximal opening 7.

In an alternative example the tubular body has a triangular tube shape with a triangular opening on the proximal end, namely the proximal opening, and optionally another triangular opening on the distal end, namely the distal opening. In other words, the tubular body has a triangular cross section and a hollow triangular tube shape.

In an alternative example the tubular body has a hexagonal tube shape with a hexagonal opening on the proximal end, namely the proximal opening, and optionally another hexagonal opening on the distal end, namely the distal opening. In other words, the tubular body has a hexagonal cross section and a hollow hexagonal tube shape.

In an alternative example the tubular body has an octagonal tube shape with an octagonal opening on the proximal end, namely the proximal opening, and optionally another octagonal opening on the distal end, namely the distal opening. In other words, the tubular body has an octagonal cross section and a hollow octagonal tube shape.

In an alternative example the tubular body has a rhombic tube shape with a rhombic opening on the proximal end, namely the proximal opening, and optionally another rhombic opening on the distal end, namely the distal opening. In other words, the tubular body has a rhombic cross section and a hollow rhombic tube shape.

The tubular body 4 has a distal end and a proximal end with openings 7, 8, wherein the proximal opening 7 is configured to receive the proximal end of the medicament delivery device 6. The tubular body 4 forms a sleeve around the proximal end of the device 6 having the functionality of a cap, therefore the opening 8 on the distal end is slightly narrower than the opening 7, the opening comprises an inner rim, not shown in the figures, in order to provide a shoulder for the proximal end of the medicament delivery device 6 to abut on. Thus, the medicament delivery device cannot pass, or fall through the distal opening 8. Alternatively, the tubular body 4 has only one opening, namely the proximal opening 7, at the distal end, and the cap has a closed end, namely the second lid (3).

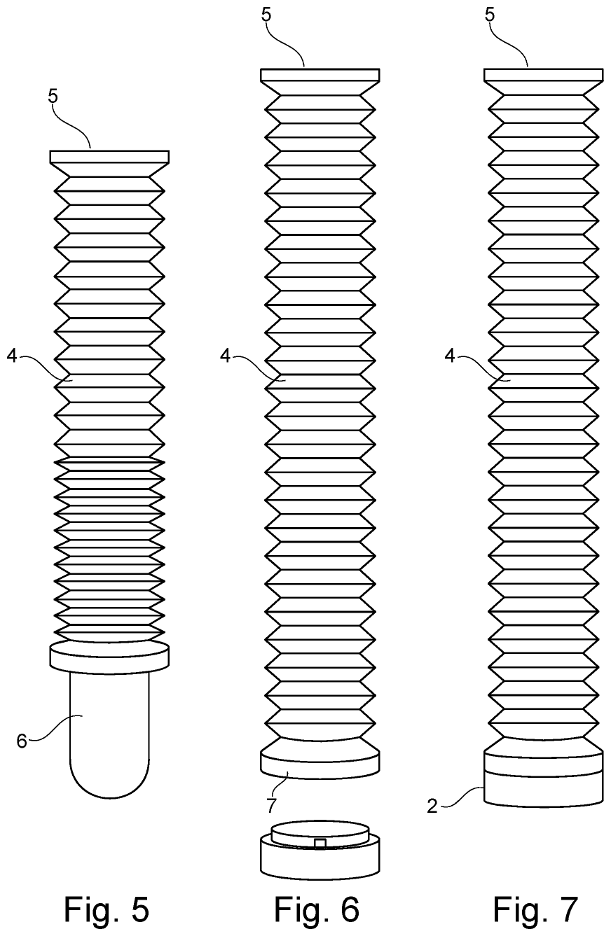

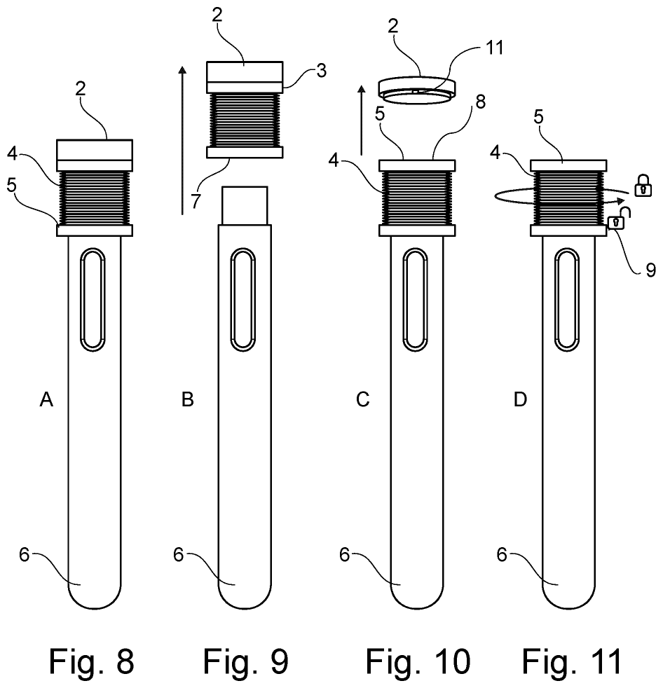

FIG. 8 shows the cap assembly 1 in the initial state A providing a cap for the medicament delivery device 6. FIG. 9 shows the cap assembly 1 taken off the medicament device 6, then as shown in FIG. 10 the first lid 2 is taken away from the tubular body 4. Then as shown in FIG. 11 the tubular body is turned to unlock the locking mechanism 9 of the tubular body 4 such that it can expand along the axis L and enclose the medicament delivery device 6 as is illustrated in FIG. 5.

In one example the tubular body 4 is expanded manually after being unlocked by pulling the second ring 5b towards the distal end of the medicament delivery device until the tubular body 4 is fully expanded.

In another example the tubular body 4 further comprises a resilient member, for example a spring, which allows a spring-assisted deployment of the tubular body into a fully extended state. Alternatively, the tubular body 4 is made of a material which is itself resilient, whereby the extended state is a default state and when the tubular body is compressed into a compact state and locked in this compact state with the locking mechanism. After unlocking the locking mechanism, the tubular body is expanded by the resilient force of the material expanding into its default state.

An advantage of the resilient member is for example that the medicament delivery device can be stored for packaging and transport in the tubular body when expanded. The user compresses the tubular body 4 before use. After use the tubular body is extended again for storing the used and disposable medicament delivery device.

FIG. 6 shows the tubular body 4 in a fully expanded state, enclosing the medicament delivery device 6, whereby the first lid 2 is not yet attached to the proximal opening 7 of the tubular body 4. Whereby the proximal opening 7 further comprises locking mechanism 11 configured to attach and lock the first lid 2 to the tubular body 4.

FIG. 7 illustrates the sharps container in its final state B with the first lid 2 attached to the proximal end of the tubular body 4.

Alternatively, the tubular body 4 has a second, closing, lid 3, configured to prevent the medicament delivery device 6 to pass or fall through the hollow tubular body of the tubular body 4, having the same functionality as the rim. The second lid 3 further comprises a mounting means for the first lid 2, in order to detachably attach the first lid 2 to the second lid 3.

In this example, as shown in FIG. 4 the tubular body 4 is fully expanded, comprising the disposable medicament delivery device 6. Whereby the medicament delivery device 6 abuts on one end the second lid 3 which prevents the device from falling out, i.e. passing through the opening 7.

In another example the tubular body 4 may have any suitable shape fitting onto the medicament delivery device, for example an elliptical, a square, a rectangular or any free form shape adapted to the specific medicament delivery device.

In one example the tubular body 4 has a folded accordion or bellows like structure, or a rolled-up structure, or even an interlocked or nested structure.

The locking mechanism is configured to hold the tubular body in a retracted state, it can be any suitable mechanism for that purpose. For example, in the form of a longitudinal seal extending from the first ring 5a to the second ring 5b, the seal is broken when the second ring 5b is twisted. This is an example of an irreversible locking mechanism, since the seal is broken only once.

In the example shown in FIGS. 12 and 13, the tubular body 4 has one opening, namely the proximal opening 7 with a first ring 5a configured to receive the medicament delivery device. At the distal end, the second ring 5b comprises a slit 91; the slit extends circumferentially adjacent to the edge of the second ring 5b. An arm 93 extends through the first ring 5a; the arm extends longitudinally from the first ring 5a to the second ring 5b. The arm 93 has a distal end and a proximal end, and the proximal end is attached to the first ring 5a. The distal end of the arm comprises a protrusion 92; the protrusion 92 is configured to snugly fit into slit 91. The protrusion follows the arc shape of the slit 91. The protrusion 92 extends beyond the slit in the distal direction. The protrusion also extends circumferentially beyond the circumferential width of the arm 93, thereby defining a circumferentially extending part. The circumferentially extending part follows the arc shape of the second ring 5b in the circumferential direction. When the first ring 5a is turned in a first direction relative to the slit 91, wherein the first direction is the direction of the circumferentially extending part of the protrusion 92, then the circumferentially extending part moves beyond the opening of the slit 91, thereby locking the tubular body. In this position the protrusion cannot pass through the slit and move in a proximal direction, thus the movement of the first ring 5a in the proximal direction is inhibited. When the first ring 5a is moved in the opposite direction to the first direction, then the protrusion with the circumferentially extending part can again align with the slit 91 such that the protrusion can pass through the slit, thereby allowing a proximal movement of the first ring 5a relative to the protrusion 92, thereby allowing the tubular body to extend.

When subsequently compressing the tubular body, the protrusion 92 with the circumferentially extending part is received by the slit 91, and when subsequently the first ring 5a is turned in the first direction, the circumferentially extending part of the protrusion moves in the circumferential direction relative to the slit so that the protrusion is beyond the opening of the slit. As a result, the first ring 5a inhibits the proximal movement of the tubular body. This provides a reversible locking mechanism.

The above described locking mechanism is covered on the distal end with a second, mounting, lid 3 which covers and closes the distal end; the second lid optionally is fixedly joined to the second ring 5b; alternatively, the second ring is removably attached to the second ring. The second lid comprises mounting means, for example threads, snap fit attachments, magnetic fasteners or any other suitable means for reversibly attaching the first lid 2 to the second lid 3.

Various modifications to the embodiments described are possible and will occur to those skilled in the art without departing from the invention which is defined by the following claims.

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

As used herein, apparatus, element and method “configured to” perform a specified function is indeed capable of performing the specified function without any alteration, rather than merely having potential to perform the specified function after further modification. In other words, the apparatus, element, and method “configured to” perform a specified function is specifically selected, created, implemented, utilized, programmed, and/or designed for the purpose of performing the specified function. As used herein, “configured to” refers to existing characteristics of an apparatus, element, and method which enable the apparatus, element, and method to perform the specified function without further modification. For purposes of this disclosure, an apparatus, element, and method described as being “configured to” perform a particular function can additionally or alternatively be described as being “adapted to” and/or as being “operative to” perform that function.

Further, in the following description, the wording medicament delivery device will be used. In this context, medicament delivery devices may include a number of devices capable of delivering certain doses of medicament to a user. The medicament delivery devices may be of either disposable type or re-usable type and may be provided with medicament containers suitably arranged for specific

It will be appreciated that other arrangements are possible as well, including some arrangements that involve more or fewer steps than those described above, or steps in a different order than those described above.

While various aspects and embodiments have been disclosed herein, other aspects and embodiments will be apparent to those skilled in the art. All embodiments within and between different aspects of the devices and methods can be combined unless the context clearly dictates otherwise. The various aspects and embodiments disclosed herein are for purposes of illustration and are not intended to be limiting, with the true scope and spirit being indicated by the claims.

1-15. (canceled)

16. A cap assembly for a medicament delivery device, the cap assembly comprising:
a tubular body; and
a first lid,
wherein the tubular body comprises a proximal opening for receiving a proximal part of a medicament delivery device,
wherein the tubular body is expandable in a longitudinal direction, and
wherein the cap assembly is configured to change shape from a first, not expanded, configuration for protecting a medicament delivery member of the medicament delivery device prior to use to a second, expanded, configuration for storing the medicament delivery device after use.

17. The cap assembly of claim 16, further comprising a second lid which is arranged, when in the first configuration, between the tubular body and the first lid.

18. The cap assembly of claim 17, wherein the tubular body comprises a distal opening, and at a distal end of the cap assembly a first ring configured to fasten the second lid, and wherein the tubular body further comprises at a proximal end a second ring.

19. The cap assembly of claim 18, wherein the tubular body is a cap for the medicament delivery device, wherein the tubular body forms a sleeve around a proximal end of the medicament delivery device, wherein the distal opening has a smaller diameter than the proximal opening, and wherein the distal opening further comprises an inner rim providing an abutting shoulder for the distal end of the medicament delivery device.

20. The cap assembly of claim 18, wherein the second ring further comprises a locking mechanism configured to lock the tubular body in a retracted state when in the first configuration.

21. The cap assembly of claim 20, wherein when the locking mechanism is unlocked the tubular body is configured to expand along an axis so as to enclose the medicament delivery device inside the tubular body.

22. The cap assembly of claim 20, wherein the locking mechanism comprises a longitudinal seal, which seal is extending from the first ring to the second ring, wherein the seal is broken when the locking mechanism is actuated.

23. The cap assembly (1) of claim 21, wherein the locking mechanism comprises a slit arranged on the second ring, wherein the slit is configured to receive a protrusion, wherein the protrusion is attached to an arm, which arm is attached to the first ring and the arm extends from the first ring to the second ring.

24. The cap assembly of claim 23, wherein the protrusion extends beyond the slit in the distal direction, the protrusion also extends circumferentially beyond a circumferential width of the arm, thereby the protrusion defines a circumferentially extending part, wherein the circumferentially extending part of the protrusion is configured to move beyond the opening of the slit when the first ring is moved in a first direction, so that the circumferentially extending part of the protrusion inhibits a movement in the proximal direction of the tubular body when the tubular body is locked in a contracted state.

25. The cap assembly of claim 24, wherein when the first ring being moved in a second direction, the second direction being opposite to the first direction, then the circumferentially extending part of the protrusion is configured to align with the slit such that the protrusion and the circumferentially part passes through the slit, such that the protrusion can move in the proximal direction, thereby allowing the tubular body to move in the proximal direction.

26. The cap assembly of claim 16, wherein the tubular body comprises an expandable structure in the form of a folded accordion, or a rolled-up structure, or an interlocked nested structure.

27. The cap assembly of claim 16, wherein the tubular body has a cross sectional shape which snugly fits the shape of the medicament delivery device which is introduced into the tubular body.

28. A method for transforming the cap assembly of claim 16 comprising the steps of:
removing the cap assembly from the medicament delivery device;
removing the first lid;
inserting the medicament delivery device into a proximal opening after the medicament delivery device has been used;
unlocking a locking mechanism;
expanding the tubular body over the whole length of the medicament delivery device; and
closing the proximal opening with the first lid.

29. The method of claim 28, further comprising the steps of:
removing a second lid; and
closing a distal opening with the second lid.

30. A medicament delivery device comprising the cap assembly according to claim 16.
